(19) United States
(12) Patent Application Publication
(10) Pub. No.: US 2025/0268845 A1
(43) Pub. Date: Aug. 28, 2025

(54) BONE REGENERATION COMPOSITION INCLUDING HORDENINE AND USE THEREOF

(71) Applicant: NEWPATHBIO INC., Seongnam-si Gyeonggi-do (KR)

(72) Inventors: Soo Young LEE, Seoul (KR); Ji Hee KIM, Seongnam-si Gyeonggi-do (KR)

(73) Assignee: NEWPATHBIO INC., Seongnam-si Gyeonggi-do (KR)

(21) Appl. No.: 19/205,328

(22) Filed: May 12, 2025

Related U.S. Application Data
(63) Continuation of application No. PCT/KR2024/002426, filed on Feb. 26, 2024.

(30) Foreign Application Priority Data
Feb. 28, 2023 (KR) 10-2023-0027336

(51) Int. Cl. A61K 31/137; A61P 19/00; A61P 19/02

(52) U.S. Cl. CPC A61K 31/137; A61P 19/00; A61P 19/02

(57) ABSTRACT

The present disclosure relates to a composition for bone regeneration and use thereof. The composition according to an aspect has the effects of regenerating cartilage and inhibiting bone resorption by osteoclasts, and thus can be effectively used for the prevention, amelioration or treatment of bone diseases.

### CROSS-REFERENCE TO RELATED APPLICATIONS

This application is a continuation of PCT/KR2024/002426 filed on Feb. 26, 2024, which claims priority to Korean Patent Application No. 10-2023-0027336 filed on Feb. 28, 2023, the entire contents of which are herein incorporated by reference.

### TECHNICAL FIELD

The present disclosure relates to a composition for bone regeneration and use thereof.

### BACKGROUND ART

Bone modeling and remodeling processes play an important role in bone development, growth and metabolism. Bone modeling begins in fetal life and continues until young adulthood when the skeleton matures and growth ends, and the maximum bone mass is formed in the 20s and early 30s. Then, for about 30 years, bone remodeling, which involves bone removal and bone supplementation, is repeated, and at this time, bone modeling and bone resorption are paired and balanced. After this period, bone modeling cannot sufficiently compensate for bone loss due to bone resorption, resulting in a decrease in bone mass of about 0.3 to 0.5% per year, and particularly, women experience a considerable bone loss of 2-3% per year in the early stage of menopause.

Bone tissue constitutes the skeletal system along with cartilage, plays a role in mechanical support and muscle attachment, protects living organs and bone marrow, and is responsible for preserving calcium and phosphorus ions to maintain the homeostasis thereof. Bone tissue consists of cell substrates such as collagen and glycoproteins, and various types of cells, including osteoblasts, osteoclasts, and bone cells.

Osteoclasts differentiate from hematopoietic stem cells in the bone marrow and increase the expression of tartrate-resistant acid phosphatase (TRAP) and Cathepsin K (Cath K), which directly act on bone resorption. In this process, there is an accompanying increase in the expression of transcriptional regulatory factors such as c-fos and NFATc1, which help the expression of osteoclast genes, and there is also an increase in the expression of receptor activator of nuclear factor KB (RANK) genes, which receive osteoclast differentiation signals from outside a cell. These genes have the characteristic of increasing as osteoclast differentiation progresses, and are therefore also used as indicators of osteoclast differentiation.

In addition, osteoclasts cause abnormal destruction and resorption of bone tissue due to an imbalance with osteoblasts in the bone, and thus are known to be the cause of osteoporosis in which bone mass and bone density decrease, osteomalacia in which calcium is depleted from the bone, fibrous dysplasia in which normal bone tissue is replaced by fibrous connective tissue and immature bone trabeculae, and rheumatoid arthritis which leads to the destruction and deformation of joints. As such, there is a close relationship between osteoclast differentiation and bone diseases.

Meanwhile, hordenine may be denoted as N,N-dimethyltyramine, and has an IUPAC name of 4-(2-dimethylaminoethyl) phenol, and may be represented by a structure of Formula 1:

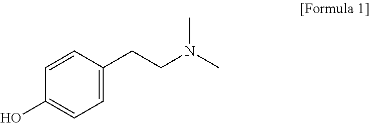

Hordenine is known to be used as a central nervous system stimulant or a nutritional supplement, or to have weight loss effects, but effects thereof on bone diseases have not been known at all.

Therefore, the inventors of the present disclosure confirmed that hordenine is effective for bone diseases by regenerating cartilage and regulating osteoclasts, thereby completing the present disclosure.

### DISCLOSURE OF INVENTION

### Technical Problem

An aspect is to provide a composition for bone regeneration, including hordenine or a pharmaceutically acceptable salt thereof.

Another aspect is to provide a pharmaceutical composition for preventing or treating a bone disease, including hordenine or a pharmaceutically acceptable salt thereof.

Another aspect is to provide a health functional food for preventing or ameliorating a bone disease, including hordenine or a sitologically acceptable salt thereof.

Another aspect is to provide a feed composition for preventing or ameliorating a bone disease, including hordenine or a sitologically acceptable salt thereof.

Another aspect is to provide a method of preventing or treating a bone disease, including administering hordenine or a pharmaceutically acceptable salt thereof to a subject in need thereof.

Another aspect is to provide use of hordenine or a pharmaceutically acceptable salt thereof for preparing a therapeutic drug for the prevention or treatment of a bone disease.

### Solution to Problem

An aspect provides a composition for bone regeneration, including hordenine or a pharmaceutically acceptable salt thereof.

In an embodiment, the hordenine or a pharmaceutically acceptable salt thereof may promote differentiation of mesenchymal stem cells into chondrocytes. Thus, the hordenine or a pharmaceutically acceptable salt thereof may regenerate cartilage.

In other embodiments, the hordenine or a pharmaceutically acceptable salt thereof may inhibit bone resorption by osteoclasts.

Therefore, the hordenine may regenerate cartilage by promoting differentiation of mesenchymal stem cells into chondrocytes and inhibit bone resorption by osteoclasts, and thus may be used for the prevention or treatment of a bone disease.

In an embodiment, the composition may further include an osteoinductive material, an osteoconductive material, an osteogenic material, an osteopromotive material, an anti-osteoporotic material, or an osteophilic material.

Specifically, “osteoinductive material” refers to a material that induces mitogenesis of undifferentiated perivascular mesenchymal cells, leading to the formation of osteoprogenitor cells (i.e., cells capable of forming new bone or bone material). “Osteoconductive material” refers to a material that facilitates blood vessel incursion and new bone or bone material formation into a predetermined passive trellis structure. These materials may include various compounds, minerals, proteins, and the like that are known to exhibit osteoinductive, osteoconductive, osteogenic, osteopromotive or osteophilic activities.

Specifically, the osteoinductive or osteoconductive material may include, but are not limited to, demineralized bone matrix (DBM), bone morphogenetic proteins (BMPs), transforming growth factors (TGFs), fibroblast growth factors (FGFs), insulin-like growth factors (IGFs), platelet-derived growth factors (PDGFs), epidermal growth factors (EGFs), vascular endothelial growth factors (VEGFs), peptides, inorganic bone mineral (ABM), vascular permeability factors (VPFs), cell adhesion molecules (CAMs), calcium aluminate, hydroxyapatite, coralline hydroxyapatite, alumina, zirconia, aluminum silicates, calcium phosphate, tricalcium phosphate, brushite (dicalcium phosphate dihydrate), tetracalcium phosphate, octacalcium phosphate, calcium sulfate, polypropylene fumarate, pyrolytic carbon, bioactive glass, porous titanium, porous nickel-titanium alloy, p7orous tantalum, sintered cobalt-chromium beads, ceramics, collagen, autologous bone, allogenic bone, xenogenic bone, coralline and derivatives or combinations thereof, or other biologically produced composite materials containing calcium or hydroxyapatite structural elements.

Specifically, the osteogenic material may include, but is not limited to, osteogenic proteins (e.g., OP-1, OP-2, or OP-3), transforming growth factor-α, transforming growth factor-β (e.g., B1, B2, or 3), LIM mineralization proteins (LMPs), osteoid-inducing factor (OIF), angiogenin, endothelins, growth differentiation factors (GDFs), ADMP-1, endothelins, hepatocyte growth factor and keratinocyte growth factor, osteogenin (bone morphogenetic protein-3), heparin-binding growth factors (HBGFs) (e.g., HBGF-1 and HBGF-2), interleukins (ILs) including IL-1 to IL-6, colony-stimulating factors (CSFs) including CSF-1, G-CSF, and GM-CSF, epidermal growth factors (EGFs), insulin-like growth factors (e.g., IGF-I and -II), demineralized bone matrix (DBM), cytokines, osteopontin, and osteonectin.

The composition may further include an additive to adjust the properties of the prepared composition. Specifically, the additive may include proteins, radiopaque agents, such as strontium phosphate or strontium oxide, drugs, supportive or reinforcing filler materials, crystal growth modifiers, viscosity modifiers, pore-forming agents, antibiotics, preservatives, growth factors, chemotherapeutic agents, bone resorption inhibitors, colorants, immersion liquids, carboxylates, carboxylic acids, α-hydroxy acids, metal ions, or mixtures thereof. As other examples, the additive may include materials that adjust coagulation time (e.g., pyrophosphate or sulfate), or increase injectability or cohesiveness (e.g., hydrophobic polymers such as collagen).

The term “pharmaceutically acceptable” as used herein means exhibiting properties that are non-toxic to cells or humans exposed to the composition.

The term “pharmaceutically acceptable salt” as used herein refers to a salt prepared from a specific compound according to an aspect and a relatively non-toxic acid or base. In case that the compound contains a relatively acidic functional group, a base addition salt may be obtained by bringing a sufficient amount of a base into contact with a neutral form of such a compound in a pure solution or a suitable inert solvent. Pharmaceutically acceptable base addition salts include salts of sodium, potassium, calcium, ammonium, organic amines, or magnesium, or similar salts. In case that the compound contains a relatively basic functional group, an acid addition salt may be obtained by bringing a sufficient amount of an acid into contact with a neutral form of such a compound, in a pure solution or a suitable inert solvent. Pharmaceutically acceptable acid addition salts include: salts of inorganic acids such as hydrochloric acid, hydrobromic acid, nitric acid, carbonic acid, bicarbonate ions, phosphoric acid, monohydrogen phosphate ions, dihydrogen phosphate ions, sulfuric acid, hydrogen sulfate ions, hydroiodic acid or phosphorous acid; and salts of organic acids such as acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-tolylsulfonic acid, citric acid, tartaric acid, and methanesulfonic acid, and further include salts of amino acids (e.g., arginine and the like) and salts of organic acids such as glucuronic acid.

The pharmaceutically acceptable salts may be synthesized by conventional chemical methods from parent compounds containing an acidic or basic moiety. Generally, these salts are prepared by reacting a free acid or base form of these compounds with a stoichiometrically suitable amount of a base or acid in water or an organic solvent or a mixture of the two. Generally, a non-aqueous medium such as ether, ethyl acetate, ethanol, isopropanol, or acetonitrile is preferable.

Another aspect provides a pharmaceutical composition for preventing or treating a bone disease, including the composition.

Another aspect provides a method of preventing or treating a bone disease, including administering hordenine or a pharmaceutically acceptable salt thereof to a subject in need thereof.

In an embodiment, the bone disease may be selected from the group consisting of osteoarthritis, rheumatoid arthritis, chondromalacia patella, ankylosing spondylitis, spondyloarthropathy, gout, synovitis, osteoporosis, osteomalacia, chondromalacia, Paget's disease, osteopenia, osteopetrosis, bone atrophy, fibrous dysplasia, osteolysis, osteogenesis imperfecta, gonarthrosis, and osteonecrosis.

The term “prevention” as used herein collectively refers to partially or completely delaying or preventing the onset or recurrence of a disease, disorder, or accompanying symptoms thereof, preventing the acquisition or reacquisition of a disease or disorder, or reducing the risk of acquiring a disease or disorder. The prevention refers to all actions that inhibit or delay the occurrence of bone diseases or bone disease-related disorders, or symptoms, via administration of the composition according to the present disclosure.

The term “treatment” as used herein refers to all actions that alleviate or beneficially alter diseases, disorders, or accompanying symptoms thereof. For example, the treatment includes the inhibition, alleviation or elimination of the development of bone diseases and related diseases.

The term “pharmaceutical composition” as used herein may refer to a molecule or compound that imparts some beneficial effect when administered to a subject. The beneficial effects may include: enabling diagnostic decisions; amelioration of a disease, symptom, disorder or condition; reduction or prevention of the onset of a disease, symptom, disorder or illness; and generally responding to a disease, symptom, disorder or condition.

The subject may be a mammal, for example, a human, a cow, a horse, a pig, a dog, sheep, a goat, or a cat.

The pharmaceutical composition may be formulated as a preparation selected from the group consisting of tablets, soft or hard capsules, pills, powders, suspensions, syrups, injections, and granules.

The pharmaceutical composition may be for oral or parenteral administration. Preparations for oral administration may include tablets, soft or hard capsules, pills, powders, suspensions, syrups, injections, granules, and the like, and parenteral administration may mean administration via routes other than oral routes, such as intra-articular, rectal, intravenous, peritoneal, intramuscular, arterial, transdermal, nasal, inhalation, ocular, and subcutaneous routes. These preparations may be prepared by mixing with one or more excipients, for example, starch, calcium carbonate, sucrose or lactose, gelatin, or the like. In addition to simple excipients, lubricants such as magnesium stearate and talc may also be used. Preparations for non-oral administration may be creams, lotions, ointments, pastes, solutions, aerosols, liquid extracts, elixirs, infusions, sachets, patches, injections, or the like. In an embodiment, the composition may be administered via intra-articular injection.

The pharmaceutical composition may include conventional fillers, extenders, binders, disintegrating agents, anticoagulants, lubricants, wetting agents, pH adjusters, nutrients, vitamins, electrolytes, alginic acid and salts thereof, pectic acid and salts thereof, protective colloids, glycerin, flavoring agents, emulsifiers, preservatives, or the like.

The pharmaceutical composition may be prepared by further including one or more pharmaceutically acceptable carriers. The pharmaceutically acceptable carrier may be saline, sterile water, Ringer's solution, buffered saline, dextrose solution, maltodextrin solution, glycerol, ethanol, or a mixture of two or more of these ingredients, and, if necessary, other conventional additives such as an antioxidant, buffer, and a bacteriostatic agent may be added. In other embodiments, the pharmaceutical composition may be formulated as an injectable dosage form such as an aqueous solution, a suspension, or an emulsion, pills, capsules, granules, or tablets by further including a diluent, a dispersing agent, a surfactant, a binder, and a lubricant. Furthermore, the pharmaceutical composition may preferably be formulated according to each disease or ingredient by using an appropriate method in the art.

In other embodiments, the therapeutically effective amount and effective dose of the pharmaceutical composition may vary depending on the formulation method, administration method, administration time and/or administration route of the pharmaceutical composition. In other embodiments, the pharmaceutical composition may vary depending on various factors including the type and degree of response to be achieved via administration of the pharmaceutical composition, the type, age, body weight, general health condition, symptoms or severity of disease, gender, diet, and excretion of a subject to which the pharmaceutical composition is administered, drugs used simultaneously or at different times in the corresponding subject, and ingredients of other compositions, and similar factors well known in the medical field. Those of ordinary skill in the art can easily determine and prescribe an effective dose for the intended treatment. The pharmaceutical composition according to the present disclosure may be administered once a day or several times a day. Therefore, the dose is not intended to limit the scope of the present disclosure in any way. For example, the dose of the pharmaceutical composition may be in a range of 1 μg/kg/day to 1,000 mg/kg/day.

Another aspect provides a health functional food for preventing or ameliorating a bone disease, including hordenine or a sitologically acceptable salt thereof.

The hordenine and the effect thereof are as described above.

The term “sitologically acceptable” as used herein means exhibiting non-toxicity to cells or humans exposed to the compound.

The term “sitologically acceptable salt” as used herein refers to a salt prepared using a specific compound according to an aspect and a relatively non-toxic acid or base, and may fall within the scope as described above with regard to “salt.”

The term “amelioration” as used herein may refer to all actions that at least reduce parameters related to conditions being treated, for example, the severity of symptoms.

In the health functional food, the active ingredient may be added directly to a food or used in combination with other foods or food ingredients, and may be used appropriately according to conventional methods. A mixing amount of the active ingredient may be suitably determined according to the intended use (for prevention or amelioration). Generally, in manufacturing food or beverages, the health functional food may be added in an amount of particularly about 15 wt % or less, more particularly about 10 wt % or less, with respect to the raw material. However, in the case of long-term intake for health and hygiene purposes or health control purposes, the amount may be below the above range.

The health functional food may be formulated as one selected from the group consisting of tablets, pills, powders, granules, fine powders, capsules, and liquid formulations by further including one or more of carriers, diluents, excipients, and additives. Foods to which a compound according to an aspect may be added include various foods, powders, granules, tablets, capsules, syrups, beverages, gum, tea, vitamin complexes, health functional foods, and the like.

Specific examples of carriers, excipients, diluents, and additives may be at least one selected from the group consisting of lactose, dextrose, sucrose, sorbitol, mannitol, erythritol, starch, acacia gum, calcium phosphate, alginate, gelatin, calcium phosphate, calcium silicate, microcrystalline cellulose, polyvinylpyrrolidone, cellulose, methylcellulose, water, sugar syrup, methylhydroxy benzoate, propylhydroxy benzoate, talc, magnesium stearate, and mineral oil.

The health functional food may contain other ingredients as essential ingredients without particular limitation, in addition to the active ingredient. For example, like normal beverages, the health functional food may contain various flavoring agents or natural carbohydrates as additional ingredients. Examples of the above-described natural carbohydrates may include: conventional sugars such as monosaccharides, e.g., glucose, fructose, and the like, disaccharides, e.g., maltose, sucrose, and the like, and polysaccharides, e.g., dextrin, cyclodextrin, and the like; and sugar alcohols such as xylitol, sorbitol, erythritol, and the like. As flavoring agents other than those described above, natural flavoring agents (thaumatin and stevia extracts (e.g., rebaudioside A, glycyrrhizin, and the like)) and synthetic flavoring agents (saccharin, aspartame, and the like) may preferably be used. The ratio of the natural carbohydrates may be appropriately determined through selection by a person skilled in the art.

In addition to these additives, the health functional food according to an aspect may include various nutrients, vitamins, minerals (electrolytes), flavoring agents such as synthetic flavoring agents and natural flavoring agents, colorants and enhancers (cheese, chocolate, and the like), pectic acid and salts thereof, alginic acid and salts thereof, organic acids, protective colloidal thickeners, pH adjusters, stabilizers, preservatives, glycerin, alcohol, and carbonating agents used in carbonated beverages. These components may be used independently or in combination, and the ratio of these additives may also be appropriately selected by a person skilled in the art.

Another aspect provides a feed composition for preventing or ameliorating a bone disease, including hordenine or a sitologically acceptable salt thereof.

The hordenine and the effect thereof are as described above.

In case that the composition is used as a feed composition, the composition may be prepared as a 20 to 90% high-concentration solution or in powder or granule form. The feed composition may further include any one or more of: organic acids such as citric acid, fumaric acid, adipic acid, lactic acid, and malic acid; phosphates such as sodium phosphate, potassium phosphate, acid pyrophosphate, and polyphosphate; and natural antioxidants such as polyphenol, catechin, alpha-tocopherol, rosemary extract, vitamin C, green tea extract, licorice extract, chitosan, tannic acid, and phytic acid. The composition may be formulated in the form of a conventional feed and may include conventional feed ingredients.

The feed may further include: grains, e.g., pulverized or crushed wheat, oats, barley, corn and rice; plant-based protein feeds, e.g., feeds mainly consisting of rapeseed, soybeans, and sunflower; animal-based protein feeds, e.g., blood meal, meat meal, bone meal, and fish meal; sugars and dairy products, e.g., dry ingredients consisting of various types of milk powder and whey powder; and the like. In other embodiments, the feed may further include nutritional supplements, digestion and absorption enhancers, growth promoters, and the like.

The feed composition may be administered to an animal alone or in combination with other feed additives in an edible carrier. In other embodiments, the feed additive may be easily administered to an animal as a top dressing, by directly mixing with an animal feed, or as an oral formulation separately from the feed. In case that the feed additive is administered separately from the animal feed, the feed additive may be prepared as an immediate-release or sustained-release formulation in combination with a sitologically acceptable edible carrier, as well known in the art. The edible carrier may be solid or liquid, for example, corn starch, lactose, sucrose, bean flakes, peanut oil, olive oil, sesame oil, and propylene glycol. In case that a solid carrier is used, the feed additive may be in the form of tablets, capsules, powders, troches, or sugar-coated tablets, or a non-dispersed top dressing. In case that a liquid carrier is used, the feed additive may be in the form of gelatin soft capsules, or formulations such as syrups, suspensions, emulsions, or solutions.

In other embodiments, the feed may contain adjuvants, e.g., preservatives, stabilizers, wetting agents or emulsifiers, solubilizing agents, and the like. The feed additive may be added to animal feed by immersion, spraying, or mixing and be used.

The feed or feed additive of the present disclosure may be applied to many animal feeds including mammals, poultry and fish.

The feed or the feed additive may be used for pigs, cows, sheep, goats, experimental rodents, and pets (e.g., dogs and cats) in addition to the experimental rodents, and the like, as the mammals, and may be used for chickens, turkeys, ducks, geese, pheasants, quails, and the like, as the poultry, and may be used for trout and the like as the fish. However, the present disclosure is not limited thereto.

### Advantageous Effects of Invention

A composition according to an aspect has the effects of regenerating cartilage and inhibiting bone resorption by osteoclasts, and thus can be effectively used for preventing, ameliorating or treating a bone disease.

### BRIEF DESCRIPTION OF DRAWINGS

FIG. 1 is an image confirming differentiation of mesenchymal stem cells into chondrocytes upon treatment with hordenine.

FIG. 2 is a view illustrating an experimental process to confirm whether hordenine is effective in osteoarthritis.

FIG. 3A shows cartilage images confirming the therapeutic effect of hordenine treatment on osteoarthritis.

FIG. 3B illustrates graphs showing OARSI grade, osteophyte maturity, subchondral bone plate (SBP) thickness, and synovitis, upon treatment with hordenine.

FIG. 4 illustrates an experimental process to confirm the effect of hordenine on osteoclasts.

FIG. 5A illustrates images and a graph, showing the inhibition of bone resorption by osteoclasts upon treatment with hordenine.

FIG. 5B illustrates images and a graph, showing the inhibition of actin ring formation by osteoclasts upon treatment with hordenine.

FIG. 6 illustrates mouse skull images and a graph, confirming the inhibition of bone resorption by osteoclasts upon treatment with hordenine, through TRAP staining.

### DETAILED DESCRIPTION

Hereinafter, preferred examples will be described to aid in understanding of the present disclosure. However, these examples are provided only to facilitate the understanding of the present disclosure and are not intended to limit the content of the present disclosure. Various modifications can be made to embodiments, and thus embodiments are not limited to the examples as set forth below, but can be embodied in various forms.

### Example 1. Confirmation of Promotion of Differentiation from Mesenchymal Stem Cells into Chondrocytes

It was investigated as follows whether hordenine affects differentiation from mesenchymal stem cells into chondrocytes. Hordenine was purchased and used, and was also used in the following examples (Sigma Aldrich 04476, Santa Cruz CAS 539-15-1).

Specifically, mesenchymal stem cells were seeded at 2.5×10⁴ cells/48 well and divided into a group treated only with the basic medium α-MEM, a group treated only with a differentiation medium (Chondrogenic media (CM)), and groups treated with 5 or 10 UM hordenine. After a few days, the degree of chondrocyte differentiation was evaluated by Alcian blue staining.

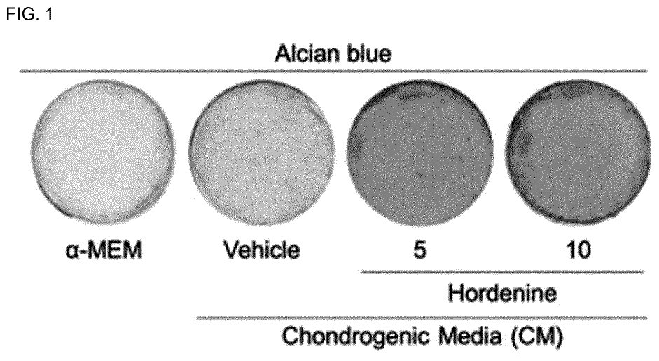

As a result, as illustrated in FIG. 1, it was confirmed that the degree of differentiation from mesenchymal stem cells into chondrocytes was much higher in the group treated with hordenine compared to the group treated only with a differentiation medium.

These results indicate that hordenine according to an embodiment promotes chondrogenic differentiation, and thus can be effectively used for the treatment of osteoarthritis.

### Example 2. Confirmation of Cartilage Regeneration Effect

The cartilage regeneration effect of hordenine on osteoarthritis was investigated as follows.

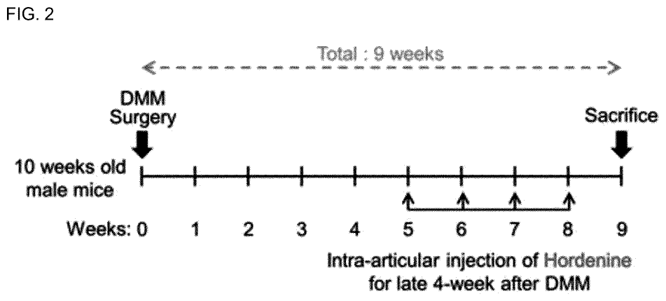

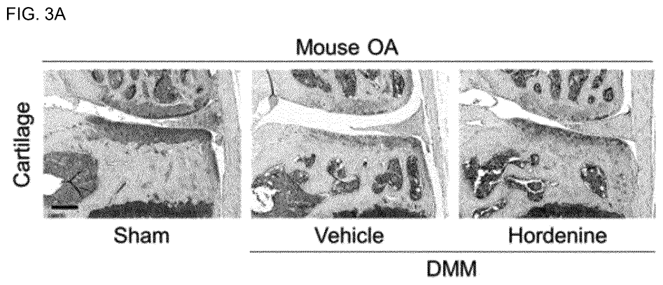

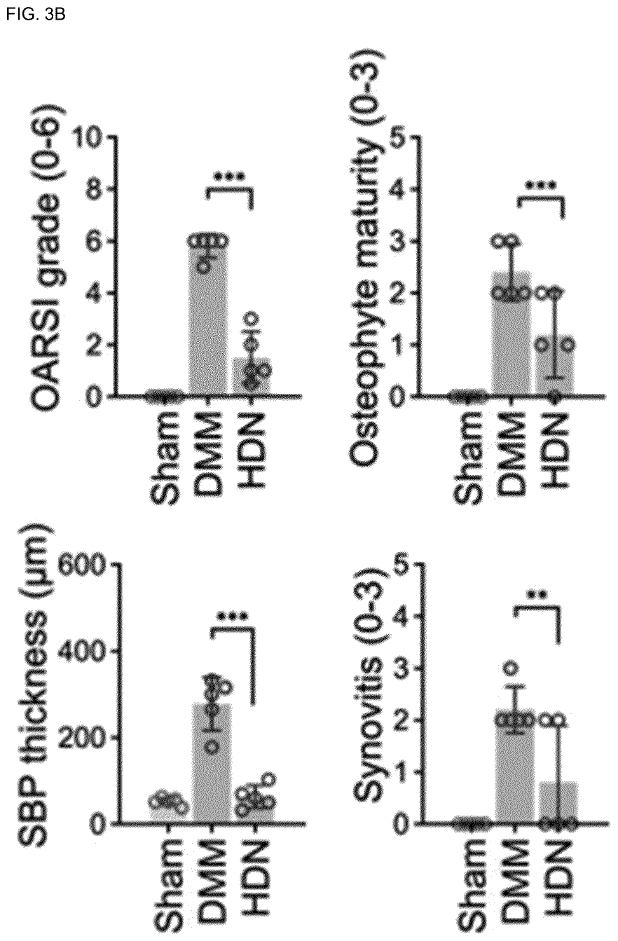

Specifically, osteoarthritis was induced by incising the ligament that stabilizes the medial meniscus in the joint tissue of 9-week-old mice. Starting from week 5 after destabilization of the medial meniscus (DMM), 2 mg/kg of hordenine was injected intra-articularly for 4 weeks, and at week 9, the mice were sacrificed and the joint tissues were examined by staining with Safranin O and fast green. The experimental process is illustrated in FIG. 2, and the experimental results are illustrated in FIGS. 3A and 3B.

As illustrated in FIGS. 3A and 3B, it was confirmed that cartilage (red portion) that had collapsed due to DMM was restored after hordenine injection. As illustrated in FIG. 2B, it was also confirmed that, after hordenine injection, osteoarthritis induced by DMM showed a decrease in cartilage degradation (OARSI grade), osteophytes caused by osteoarthritis induction were also reduced, the thickness of subchondral bone plate (SBP) was reduced, and synovitis was reduced.

These results indicate that hordenine prevents the progression of osteoarthritis and regenerates cartilage, and thus has the effects of preventing and treating osteoarthritis.

### Example 3. Confirmation of Inhibitory Effect on Osteoclast Function

### 3.1. In Vitro Confirmation of Inhibitory Effect on Osteoclast Function

To confirm whether hordenine has the effect of inhibiting osteoclast function, bone resorption analysis and actin ring analysis were performed as follows.

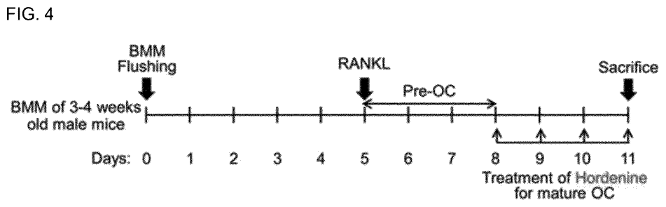

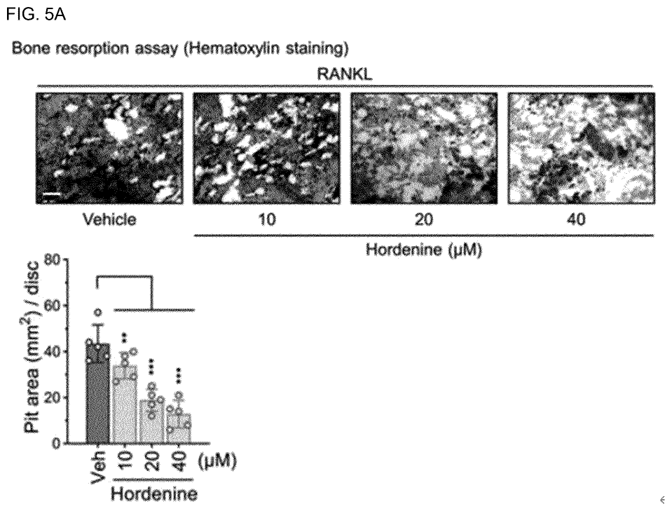

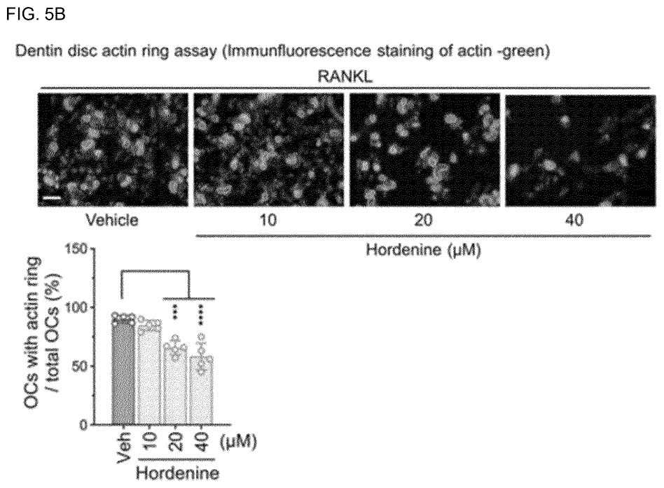

Specifically, bone marrow-derived macrophages (BMMs) were isolated from 3- to 4-week-old mice and cultured. On day 5, the BMMs were treated with receptor activator of nuclear factors KB ligand (RANKL) and macrophage-colony stimulating factor (M-CSF) for 3 days to be differentiated into pre-osteoclasts. Thereafter, to confirm the degree of osteoclast activation, the pre-osteoclasts were treated with 10, 20, or 40 μM of hordenine for 3 days, and a bone resorption assay through hematoxylin staining and a dentin disc actin ring assay through immunofluorescence staining were performed to confirm the degree of osteoclast activation. Bone resorption assay is a method of seeding osteoclasts on a dentin disc, which is an experimental bone tissue simulating the dentin component of bone, and quantifying the extent to which osteoclasts eat away at the bone. When holes formed by bone erosion are quantified, the pit area is measured, and a lower measurement indicates a lower degree of bone erosion. The more extensively the disc is eroded, the more intensely the disc stains with hematoxylin, while less eroded areas stain more lightly. Actin ring formation assay is an analysis that determines how much actin ring formation is inhibited, as osteoclasts form actin rings as a prerequisite for bone resorption and inhibition of actin ring formation indicates the inhibition of differentiation of osteoclasts to a stage capable of bone resorption. The experimental process is illustrated in FIG. 4, and the experimental results are illustrated in FIGS. 5A and 5B.

As illustrated in FIG. 5A, it was confirmed that hematoxylin staining became lighter in a concentration-dependent manner and the pit area was reduced, upon treatment with hordenine, compared to non-treatment with hordenine. As illustrated in FIG. 5B, it was also confirmed that the number of osteoclasts forming actin rings decreased in a concentration-dependent manner upon treatment with hordenine, compared to non-treatment with hordenine.

These results indicate that hordenine inhibits bone resorption by osteoclasts, and thus can be used for the prevention or treatment of bone diseases.

### 3.2. In Vivo Confirmation of Inhibitory Effect on Osteoclast Function

Mouse skulls were examined as follows to confirm whether hordenine has the effect of inhibiting osteoclast function.

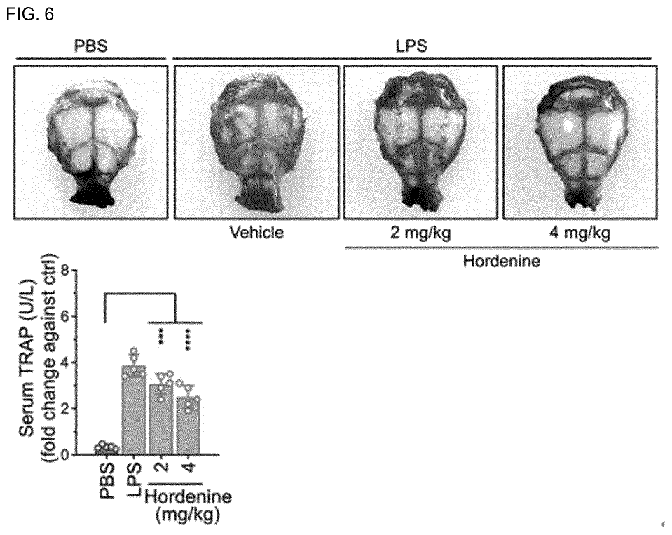

Specifically, 12.5 mg/kg of lipopolysaccharide (LPS) was injected intra-calvarially into the skull of 5-week-old male mice twice in total: once on the first injection day and once 2 days after the first injection day, thereby inducing bone destruction. Thereafter, 1 mg/kg of hordenine was injected intra-calvarially for 5 days, starting 1 day after the first LPS injection day. 6 days after the first LPS injection day, the mice were sacrificed and the skulls were harvested from the mice. Then, the degree of osteoclast differentiation was evaluated by tartrate-resistant acid phosphatase (TRAP) staining. The results thereof are illustrated in FIG. 6.

As illustrated in FIG. 6, it was confirmed that the skulls turned red by TRAP staining due to the activation of osteoclasts upon treatment with LPS, whereas the skulls were lightly stained with TRAP upon treatment with hordenine.

These results indicate that hordenine inhibits bone resorption by suppressing the activity of osteoclasts, and thus can be used for the prevention or treatment of bone diseases.

1. A composition for bone regeneration, comprising hordenine or a pharmaceutically acceptable salt thereof.

2. The composition of claim 1, wherein the hordenine or a pharmaceutically acceptable salt thereof promotes differentiation of mesenchymal stem cells into chondrocytes.

3. The composition of claim 1, wherein the hordenine or a pharmaceutically acceptable salt thereof regenerates cartilage.

4. The composition of claim 1, wherein the hordenine or a pharmaceutically acceptable salt thereof inhibits bone resorption by osteoclasts.

5. The composition of claim 1, further comprising one or more selected from the group consisting of an osteoinductive material, an osteoconductive material, an osteogenic material, an osteopromotive material, an anti-osteoporotic material, and an osteophilic material.

6. A method of preventing or treating a bone disease, comprising administering hordenine or a pharmaceutically acceptable salt thereof to a subject in need thereof.

7. The method of claim 6, wherein the bone disease is one or more selected from the group consisting of osteoarthritis, rheumatoid arthritis, chondromalacia patella, ankylosing spondylitis, spondyloarthropathy, gout, synovitis, osteoporosis, osteomalacia, chondromalacia, Paget's disease, osteopenia, osteopetrosis, bone atrophy, fibrous dysplasia, osteolysis, osteogenesis imperfecta, gonarthrosis, and osteonecrosis.

8. The method of claim 6, wherein the hordenine or a pharmaceutically acceptable salt thereof is administered via intra-articular injection.

9. A health functional food for preventing or ameliorating a bone disease, comprising hordenine or a sitologically acceptable salt thereof.
